(19) United States
(12) Patent Application Publication
(10) Pub. No.: US 2007/0065824 A1
(43) Pub. Date: Mar. 22, 2007

(54) Method for the detection of cytosine methylations in dna by means of cytidine deaminases

(76) Inventor: David Gutig, Berlin (DE)

Correspondence Address: KRIEGSMAN & KRIEGSMAN, 30 TURNPIKE ROAD, SUITE 9, SOUTHBOROUGH, MA, 01772, US

(21) Appl. No.: 10/563,484

(22) PCT Filed: Jun. 29, 2004
(86) PCT No.: PCT/EP04/07052

§ 371 Date: Aug. 3, 2006

(30) Foreign Application Priority Data
Jul. 4, 2003 (DE) 103 31 107.5

(51) Int. Cl. C12Q 1/68

(52) U.S. Cl. 435/6

(57) ABSTRACT

The present invention concerns a method for investigating cytosine methylations in DNA sequences. By this means, the DNA to be investigated is reacted with a cytidine deaminase which deaminates cytidine more rapidly than 5-methylcytidine. Cytosine is converted to uracil by the conversion, whereas 5-methylcytosine remains essentially unchanged. The enzymatically pretreated DNA is preferably amplified and can be analyzed by different methods. The method according to the invention is particularly suitable for the diagnosis of cancer diseases and other disorders associated with a change in the methylation status, as well as for the prognosis of undesired drug effects.

The invention concerns a method for investigating cytosine methylations in DNA sequences. By this means, the DNA to be investigated is reacted with a cytidine deaminase which deaminates cytidine more rapidly than 5-methylcytidine. Cytosine is converted to uracil by the conversion, whereas 5-methylcytosine remains essentially unchanged. The enzymatically pretreated DNA is preferably amplified and then can be analyzed by different methods. The method according to the invention is particularly suitable for the diagnosis of cancer diseases and other disorders associated with a change in the methylation status, as well as for the prognosis of undesired effects of drugs.

### BACKGROUND OF THE INVENTION

5-Methylcytosine is the most frequent covalently modified base in the DNA of eukaryotic cells. It plays an important biological role, and is involved, among other things, in the regulation of transcription, in genetic imprinting and in tumorigenesis (for review: Millar et al.: Five not four: History and significance of the fifth base. In: The Epigenome, S. Beck and A. Olek, eds.: The Epigenome. Wiley-VCH Publishers Weinheim 2003, pages 3-20). The identification of 5-methylcytosine as a component of genetic information is thus of considerable interest. It is difficult to detect methylation, of course, since cytosine and 5-methylcytosine have the same base-pairing behavior. Many of the conventional detection methods based on hybridization are thus not capable of distinguishing between cytosine and methylcytosine. In addition, the methylation information is completely lost in a PCR amplification.

The conventional methods for methylation analysis operate essentially according to two different principles. In the first one, methylation-specific restriction enzymes are utilized, and in the second one, a selective chemical conversion of unmethylated cytosines to uracil is conducted (so-called bisulfite treatment, see e.g.: DE 101 54,317 A1; DE 100 29,915 A1). The enzymatically or chemically pretreated DNA is then amplified for the most part and can be analyzed in different ways (for review: WO 02/072880, pp. 1 ff).

The conventional methods suffer from several disadvantages. The treatment with methylation-specific restriction enzymes is limited to specific sequences by the sequence specificity of the enzymes. The bisulfite treatment is time-consuming and laborious. Reaction times of more than four hours are necessary in order to achieve a complete conversion. As a result, most of the DNA, of course, is decomposed. In this case, the fraction of degraded DNA is estimated at between 84% and 96% (see: Grunau et al.: Bisulfite genomic sequencing: systematic investigation of critical experimental parameters. Nucleic Acids Res. Jul. 1, 2001;29(13)). Because of this high rate of decomposition, it is difficult to utilize the bisulfite conversion for investigations in which the DNA to be investigated is limited. A particularly interesting field of application of methylation analysis, however, lies in diagnosing cancer diseases or other disorders associated with a change in methylation status by means of DNA from body fluids, e.g. from blood or urine. However, DNA is present only in small concentrations in body fluids, so that the application of conventional bisulfite treatment is associated with difficulties.

Due to the particular biological significance of cytosine methylation and due to the above-mentioned disadvantages of the conventional methodology, there exists a great technical need for an improved and simplified method for methylation analysis. Such a method is described in the following. The invention which is disclosed here is based on the application of cytidine deaminases, which convert cytidine and 5-methylcytidine at different rates. It particularly involves activation-induced cytidine deaminase—AID. This enzyme can convert unmethylated cytosine into uracil, while methylated cytosine remains essentially unchanged. In the case of a complete conversion, a DNA sequence is thus formed in which all of the cytosines that are still present are methylated, while the originally unmethylated cytosines are now present as uracils. Consequently, the result of the enzymatic conversion corresponds to that of chemical pretreatment with bisulfite. The enzymatic method, of course, is more rapid and milder than the chemical method. Cytosines in the enzymatically pretreated DNA can be detected by means of conventional molecular biology methodology. The same methods as for the analysis of chemically pretreated DNA can be used in particular, e.g., with the application of polymerase reactions. An additional advantage of the method according to the invention thus lies in the fact that the application of detection methods is possible without a laborious purification of the DNA. The latter is necessary in the case of chemically pretreated DNA for the removal of the bisulfite.

Biologically, the AID enzyme plays an important role in antibody diversification in B cells. It particularly participates in somatic hypermutation (SIM), in gene conversion and in class switch recombination (CSR) (for review: Storb and Stavnezer: Immunoglobulin Genes: Generating Diversity with AID and UNG. Curr Biol. Oct. 29, 2002;12(21):R725-7).

Single-stranded DNA is necessary for the AID activity. AID cannot convert intact, double-stranded DNA. Of course, a cytosine deamination can produce double-stranded DNA in double-stranded regions. Thus, AID can convert single-stranded DNA that is directly transcribed. In this manner, deamination is produced in the non-template strand, which is exposed during the transcription (Chaudhuri et al., Transcription-targeted DNA deamination by the AID antibody diversification enzyme. Nature. Apr. 17, 2003; 422(6933):726-30; Ramiro et al., Transcription enhances AID-mediated cytidine deamination by exposing single-stranded DNA on the nontemplate strand. Nat Immunol. May 4, 2000(5):452-6). In the case of single-stranded DNA, a nearly 100% conversion of cytosine to uracil can be achieved within 15 minutes. Here, the activity of AID in double-stranded DNA containing partially noncomplementary sequences is in part clearly higher than in the case of single-stranded DNA. This is particularly true for noncomplementary regions with a size between five and nine nucleotides (Bransteitter et al., loc. cit. 2003, p. 4105, FIG.4a;p. 4106 Table 1). The specificity of AID for cytosine is approximately 10 times greater than that for 5-methylcytosine (Bransteitter et al., loc.cit., 2003, p. 4105 FIG. 4b;p. 4106). A technical application of these properties of the AID enzyme has not been described previously in the literature. Among other things, nucleic acid and amino acid sequences of AID are disclosed in US Patent Application US 2002-0 164,743 (=EP 1,174,509). An applicability of the enzyme to the investigation of cytosine methylations, however, is also not mentioned here. Therefore the method according to the invention, for the first time, provides access to the AID enzyme for methylation analysis. Due to the particular biological significance of cytosine methylation and due to the disadvantages of the known methods, the revealing of this new technology represents an important technical advance.

### DESCRIPTION

The method according to the invention permits detection of cytosine methylations in DNA. In this method, the DNA to be investigated is brought into contact with a cytidine deaminase, whereby the cytidine deaminase deaminates cytidine and 5-methylcytidine at different rates. The partially deaminated DNA is then investigated with respect to its sequence. Then a conclusion is made on the methylation status of the DNA to be investigated from the presence or the proportion of deaminated positions.

In a preferred embodiment, the enzyme activation-induced cytidine deaminase (AID) is used. Insofar as they are available, however, other cytidine deaminases can be used, which convert cytidine and 5-methylcytidine at different rates. The AID enzyme can be obtained in different ways. The expression of the enzyme, e.g., in insect cells is described in the literature. The enzyme obtained in this way must, of course, be reacted prior to use with an RNAse in order to remove an RNA inhibitor (Bransteitter et al. 2003, loc. cit.). The expression of AID in E. coli is also known. An RNAse treatment is not necessary here (Sohail et al. 2003, loc. cit.). Also described is an isolation of AID from stimulated, murine B-cells (Chaudhuri et al., 2003, loc. cit.) Other possibilities for producing and isolating proteins are known to the person skilled in the art. In addition to human AID, enzymes from other sources can also be used for the method according to the invention, particularly from mammals, e.g., from cows, pigs, sheep, mice, etc. It is obvious that biologically active fragments as well as modifications of the enzyme, e.g. heat-stable variants can also be used for the method according to the invention.

The DNA to be investigated may originate from different sources. For diagnostic questions, e.g., tissue samples can serve as the initial material, but body fluids, particularly serum, can also be used. It is also conceivable to use DNA from sputum, stool, urine, or cerebrospinal fluid. Preferably, the DNA is isolated from biological specimens. The DNA is extracted according to standard methods from blood, e.g., with the use of the Qiagen UltraSens DNA extraction kit. The isolated DNA can then be fragmented, e.g., by reaction with restriction enzymes. The reaction conditions and the enzymes employed are known to the person skilled in the art and result, e.g., from the protocols supplied by the manufacturers.

The DNA to be investigated must be present in single-stranded form, at least partially, for the conversion with AID. Different ways are known to the person skilled in the art for obtaining single-stranded DNA. In a preferred variant, the DNA to be investigated is heat-denatured and then hybridized with oligonucleotides which are partially complementary to the DNA to be investigated. The oligonucleotides are not directly complementary at the cytosine positions to be investigated, so that in these regions, single-stranded “bubbles” are formed, at which the AID can be active. The non-complementary regions are thus preferably between 3 and 20 nucleotides, particularly preferred between 5 and 12 nucleotides and most particularly preferred 9 nucleotides long (see Bransteitter et al., 2003, p. 4106, Table 1). In a preferred embodiment, synthetic oligonucleotides are utilized. These preferably have a length between 20 and 150, particularly preferred between 35 and 60 nucleotides. These oligonucleotides are utilized in excess relative to the DNA to be investigated, so that it is assured that as many of the cytosine positions to be investigated as possible are accessible to the deaminase. A concentration of 1 pM to 1000 nM is thus preferred, and a range between 1 nM and 100 nM is particularly preferred. In a preferred embodiment of the method according to the invention, several oligonucleotides of different sequence are utilized, so that a simultaneous an investigation of several cytosine positions is possible. In another preferred variant, the oligonucleotides are constructed in such a way that they cannot themselves be converted by the AID enzyme. This can be done, e.g., if 5-methylcytosines are contained in the oligonucleotides instead of cytosines. It is known to the person skilled in the art that other oligomers can also be utilized instead of oligonucleotides, e.g., peptide nucleic acid (PNA) oligomers. The synthesis of oligomers as well as the hybridization conditions belong to the prior art. It is obvious that instead of chemically synthesized oligonucleotides, oligonucleotides of other origin, e.g., PCR fragments or genomic DNA can be utilized also for the method according to the invention. Reaction conditions for the deamination are described in the literature (see e.g.: Bransteitter et al. 2003, loc. cit.; Sohail et al. 2003, loc. cit.; Chaudhuri et al. 2003, loc. cit.).

The converted DNA can be analyzed on the basis of familiar molecular biology methods, e.g., by means of hybridization or sequencing. In a preferred variant, the converted DNA is first amplified. The person skilled in the art knows different methods for this, e.g., ligase chain reactions. In a preferred embodiment, the DNA is amplified, of course, by means of a polymerase reaction. Different techniques are conceivable for this, e.g., the use of isothermal amplification methods. Of course, polymerase chain reactions (PCR) are particularly preferred. In a most particularly preferred embodiment, the PCR is conducted with the use of primers which bind specifically only to positions of the converted sequence that were previously either methylated (or in the reverse approach: unmethylated). This method is known under the name methylation-sensitive PCR (MSP) in the case of bisulfite-treated DNA. For this purpose, primers are used which contain at least one 5′-CpG-3′ dinucleotide, preferably primers which bear at least three 5′-CpG-3′ positions, at least one of which is localized at the 3′ end. Correspondingly, 5′-TG-3′ or 5′-CA-3′-dinucleotides are necessary for the amplification of unmethylated sequences and/or the counterstrands (see: Herman et al.: Methylation-specific PCR: a novel PCR assay for methylation status of CpG islands. Proc Natl Acad Sci U S A. Sep. 3, 1996; 93(18):9821-6).

Another particularly preferred embodiment for bisulfite-pretreated DNA is known under the name “Heavy Methyl” method. Here, a specific amplification only of the originally methylated (or unmethylated) DNA is achieved by the use of at least one methylation-specific blocker oligomer. The blocker binds to a 5′-CG-3′ (or 5′-TG-3′ dinucleotide or 5′-CA-3′) dinucleotide and thus prevents the amplification of the background DNA. Said embodiment can by realized via the selection of the polymerase or via the modification of blocker oligomers, so that a decomposition or an extension of the blocker is minimized (for review: WO 02/072880).

For the case when the necessary formation of single-stranded DNA is achieved via the use of partially complementary oligomers (see above), additional preferred embodiments are provided for the above-named “MSP” and “Heavy Methyl” variants. In this case, for the PCR amplification, at least one primer is utilized which bears the genomic sequence in the 5′ region (corresponds to the double-stranded, and thus the unconverted part of the DNA to be investigated) and which makes available in its 3′ region one sequence which corresponds to the converted DNA In the MSP variant, the 3′ region additionally bears methylation-specific positions.

The amplificates can be detected by means of conventional methods, e.g., by means of methods of length measurement such as gel electrophoresis, capillary gel electrophoresis and chromatography (e.g. HPLC). In addition, mass spectrometry and methods for sequencing such as the Sanger method, the Maxam-Gilbert method and Sequencing by Hybridisation (SBH) can be used. In a preferred embodiment, the amplificates are detected by means of primer extension methods (see, e.g.: Gonzalgo & Jones: Rapid quantitation of methylation differences at specific sites using methylation-sensitive single nucleotide primer extension (Ms-SNuPE). Nucleic Acids Res. Jun. 15, 1997;25(12):2529-31; DE 100 10,282; DE 100 10,280).

In another preferred embodiment, the amplificates are analyzed by means of hybridization to oligomer arrays (an overview of array technology can be found in the supplemental issue of: Nature Genetics Supplement, Volume 21, January 1999). The different oligomers on such an array can be arranged on a solid phase in the form of a rectangular or hexagonal grid. The solid-phase surface is preferably comprised of silicon, glass, polystyrene, aluminum, steel, iron, copper, nickel, silver, or gold. Nitrocellulose and plastics such as nylon, however, are also possible, which can exist in the form of pellets or also as a resin matrix. The, e.g., fluorescently labeled amplificates are hybridized to the bound oligomers and the unbound fragments are removed. It is thus advantageous if the oligomers hybridize to the DNA to be analyzed over a segment that is 12-22 bases long and they comprise a CG, TG or CA dinucleotide. The fluorescent signals can be scanned and can be processed with software programs (see, e.g,: Adorjan et al., Tumour class prediction and discovery by microarray-based DNA methylation analysis. Nucleic Acids Res. Mar. 1, 2002;30 (5): e21).

In another particularly preferred embodiment, the amplificates are analyzed with the use of PCR real-time variants (see: Heid et al.: Real time quantitative PCR. Genome Res. Oct. 1996;6(10):986-94, U.S. Pat. No. 6,331,393 “Methyl Light”). Here, the amplification is conducted in the presence of a fluorescently labeled reporter oligonucleotide, which hybridizes to a 5′-CG-3′ dinucleotide (or a 5′-TG-3′ or 5′-CA-3′ dinucleotide). The reporter oligonucleotide thus preferably binds to the DNA to be investigated and indicates its amplification by means of an increase or decrease in the fluorescence. Here, it is particularly advantageous if the change in fluorescence is utilized directly for the analysis and the methylation status is concluded from the fluorescent signal. A particularly preferred variant is thus the “Taqman” method. In another particularly preferred embodiment, an additional fluorescently labeled oligomer is used, which hybridizes in the direct vicinity of the first reporter oligonucleotide and this hybridization can be detected by means of fluorescence resonance energy transfer (“Lightcycler” method).

A preferred embodiment of the invention is to amplify several fragments simultaneously by means of a multiplex PCR. Care must be taken in this design that not only the primers, but also the additional oligonucleotides utilized must not be complementary to one another, since a high degree of multiplexing in this case would be more difficult than in the usual case. In the case of enzymatically pretreated DNA, however, one thus has the advantage that a forward primer can never function also as a reverse primer, due to the different G and C content of the two DNA strands, which in turn facilitates the multiplexing and the above-described disadvantage is essentially compensated for. The amplificates can be detected in turn by means of different methods. For example, the use of real-time variants is conceivable. For amplifications of more than four genes, however, it is recommended that the amplificates be detected in another way. In this case, an analysis by means of arrays (see above) is preferred.

In another preferred embodiment, after the amplification, a repeated conversion with the AID enzyme is conducted. The cytosines remaining after the first deamination are also converted in this way. Such a repeated conversion has several advantages and has been described already for bisulfite treatment (see: DE 100 50,942).

For the rest, it should be emphasized once more that the result of the enzymatic conversion according to the invention corresponds to the result of the bisulfite treatment. It is thus obvious that all of the methods already known for the analysis of bisulfite-treated DNA can also be used for the analysis of the DNA converted according to the invention. The person skilled in the art can find information on the corresponding methods in the scientific publications and in the patent literature. A current review of the possible methods is found in: Fraga and Esteller: DNA Methylation: A Profile of Methods and Applications. Biotechniques 33:632-649 (September 2002).

A particularly preferred use of the method according to the invention lies in the diagnosis of cancer diseases or other disorders associated with a change in the methylation status. These include, among others: CNS malfunctions, symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disorder of the skin, the muscles, the connective tissue or the bones; endocrine and metabolic malfunction, damage or disease; headaches or sexual malfunction. The method according to the invention is also suitable for predicting undesired drug interactions and for the differentiation of cell types or tissues or for the investigation of cell differentiation.

The invention also includes the use of cytidine deaminases which convert cytidine and 5-methylcytidine at different rates, particularly the use of activation-induced cytidine deaminase (AID), a biologically active fragment of AID or a modification thereof for methylation analysis, particularly for the diagnosis of cancer diseases or other disorders associated with a change in the methylation status, for predicting undesired drug interactions, for the differentiation of cell types and tissues or for the investigation of cell differentiation.

A kit is finally also included in the invention, which [kit] comprises the AID enzyme, a biologically active fragment of AID or a modification thereof as well as oligomers and the buffers necessary for the deamination, as well as, optionally, also a polymerase, primers and probes for an amplification and detection.

### EXAMPLE

Detection of CpG methylation in exon 1 of Homo sapiens p16-INK4 (p16) gene in human DNA (Promega)

The following sequence from the p16-INK4 gene will be investigated for its methylation status:

| | | |
|---|---|---|
| 1 | gaagaaagag gaggggctgg ctggtcacca gagggtgggg cggaccgcgt gcgctcggcg | (Seq ID 1) |
| 61 | gctgcggaga gggggagagc aggcagcggg cggcggggag cagcatggag ccggcggcgg | |
| 121 | ggagcagcat ggagccttcg gctgactggc tggccacggc cgcggcccgg ggtcgggtag | |
| 181 | aggaggtgcg ggcgctgctg gagg^{g}ggggg cgctgcccaa cgcaccgaat agttacggtc | |
| 241 | ggaggccgat ccaggtgggt agagggtctg cagcgggagc aggggatggc gggcgactct | |
| 301 | ggaggacgaa gtttgcaggg gaattggaat caggtagcgc | |

For this purpose, 160 ng of the DNA to be investigated (as a control, 160 ng of artificially methylated genomic DNA, Promega) and 25 pM of each of the two oligonucleotides

| | |
|---|---|
| 5′-ctcccaccccgcctgcgcgtgcgctcccgccgacgcctctc-3′ | (Seq ID 2) and |
| 5′-gccgactgaccgaccccacggccgcccgggccccagccca-3′ | (Seq ID 3) |

are denatured in a reaction well containing 20 μl of buffer (10 mM Tris-HC1 (pH 8.0), 1 mM EDTA, 1 mM DTT) for 5 min at 96° C. and then placed on ice for 2 min for cooling. In this way, the oligonucleotides hybridize to the genomic DNA, each with a 10 bp wide opening, which forms an optimal substrate for the subsequent treatment with the AID enzyme. For this purpose, 400 μg of AID and 2 μg of RNaseA (Bransteitter et al., PNAS, v. 100, p. 4102 (2003)) are added to the cooled reaction mixture, and the mixture is incubated for 7 min at 37° C. The reaction is terminated by a phenol/cloroform/isoamyl (25:24:1) precipitation. Detection is made by means of a PCR. For this purpose, 2 μl of the precipitated DNA solution in 18 μl of water are mixed with 2 μl of primer solution, containing 25 pM each of two oligonucleotides (5′-cgcctggcgcacgcaaa-3′ (Seq ID 4), 5′-ttacggtcggggcccgggctc-3′ (Seq ID 5)) and 2.5 μl of dNTP mix (Fermentas, concentration of 2.5 μmol/μl for each dNTP), 0.3 μl of Hot Star Taq (Qiagen), 2.5 μl of 10×PCR buffer solution (Qiagen, 15 mM MgCl₂ contained in the buffer) in a reaction well and incubated on a thermocycler with the following temperature program:
1. 95° C. 15 min
2. 95° C. 1 min
3. 55° C. 45 sec
4. 72° C. 1 min 15 sec
5. go to 2. Rep[eat] 39 [times]
6. 72° C. 10 min
7. Hold 10° C.

The PCR is monitored by gel electrophoresis. For this purpose, 5 μl of PCR product with 3 μl of Loading Dye are loaded onto a 1.4% agarose gel (Eurogentec., Inc.). 1×TBE serves as the running buffer. The fragments are stained with ethidium bromide and the gel is photographed under UV illumination.

1. A method for the detection of cytosine methylations in DNA is hereby characterized in that
a) the DNA to be investigated is brought into contact with a cytidine deaminase, whereby the cytidine deaminase deaminates cytidine and 5-methylcytidine at different rates,
b) the partially deaminated DNA is investigated with respect to its sequence, and
c) from the presence or the proportion of deaminated positions, conclusions can be made on the methylation status of the DNA to be investigated in said positions.

2. The method according to claim 1, further characterized in that activation-induced cytidine deaminase—AID or a biologically active fragment of AID or a modification thereof can be used as the methylation-specific cytidine deaminase.

3. The method according to claim 1, further characterized in that the DNA to be investigated is present at least partially in single-stranded form.

4. The method according to claim 1, further characterized in that the DNA to be investigated hybridizes with oligomers, whereby the hybrids are present in single-stranded form at the cytosine positions under investigation.

5. The method according to claim 4, further characterized in that the single-stranded regions are between 3 and 20 nucleotides long.

6. The method according to claim 4, further characterized in that the single-stranded regions are between 5 and 12 nucleotides long.

7. The method according to claim 4, further characterized in that the single-stranded region is 9 nucleotides long.

8. The method according to claim 1, further characterized in that the oligomers have a length of 20 to 150 nucleotides.

9. The method according to claim 1, further characterized in that the oligomers have a length of 35 to 60 nucleotides.

10. The method according to claim 4, further characterized in that the oligomers are present in a concentration of 1 pM to 1000 nM.

11. The method according to claim 4, further characterized in that the oligomers are present in a concentration of 1 nM to 100 nM.

12. The method according to claim 1, further characterized in that the DNA to be investigated is amplified after the enzyme treatment.

13. The method according to claim 12, further characterized in that the amplification is conducted by means of a polymerase reaction.

14. The method according to claim 13, further characterized in that the amplification is conducted by means of a polymerase chain reaction.

15. The method according to claim 14, further characterized in that the polymerase chain reaction is conducted by means of methylation-specific primers.

16. The method according to claim 14, further characterized in that at least one methylation-specific blocker oligomer is utilized in the polymerase chain reaction.

17. The method according to claim 12, further characterized in that a repeated enzymatic conversion with a cytidine deaminase is conducted after the amplification.

18. The method according to claim 12, further characterized in that the amplificates are analyzed by means of methods of length measurement, mass spectrometry or sequencing.

19. The method according to claim 12, further characterized in that the amplificates are analyzed by means of the primer extension method.

20. The method according to claim 12, further characterized in that the amplificates are analyzed by hybridization to oligomer arrays.

21. The method according to claim 12, further characterized in that the amplificates are analyzed with the use of real-time variants.

22. The method according to claim 21, further characterized in that a Taqman or a Lightcycler method is conducted.

23. The method according to claim 12, further characterized in that several fragments are simultaneously amplified by means of a multiplex reaction.

24. Use of a method according to claim 1 for the diagnosis of cancer diseases or other disorders associated with a change in the methylation status.

25. Use of a method according to claim 1 for predicting undesired drug interactions, for the differentiation of cell types and tissues or for the investigation of cell differentiation.

26. Use of cytidine deaminases, which convert cytidine and 5-methylcytidine at different rates, for methylation analysis.

27. Use of cytidine deaminases, which convert cytidine and 5-methylcytidine at different rates, for the diagnosis of cancer diseases or other disorders associated with a change in the methylation status.

28. Use of cytidine deaminases, which convert cytidine and 5-methylcytidine at different rates, for predicting undesired drug interactions, for the differentiation of cell types and tissues or for the investigation of cell differentiation.

29. Use according to claim 24, further characterized in that the cytidine deaminase involves activation-induced cytidine deaminase (AID), a biologically active fragment of AID or a modification thereof.

30. A kit, which comprises the AID enzyme, a biologically active fragment of AID or a modification thereof as well as oligomers and the buffers necessary for the deamination, as well as optionally also a polymerase, primers and probes for an amplification and detection.


SEQUENCE LISTING (5 sequences)

SEQ ID NO: 1 - DNA, 340 bp, Homo Sapiens
gaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggggggggcgctgcccaacgcaccgaatagttacggtcggaggccgatccaggtgggtagagggtctgcagcgggagcaggggatggcgggcgactctggaggacgaagtttgcaggggaattggaatcaggtagcgc

SEQ ID NO: 2 - DNA, 41 bp, Artificial Sequence
  Oligonucleotide
ctcccaccccgcctgcgcgtgcgctcccgccgacgcctctc

SEQ ID NO: 3 - DNA, 40 bp, Artificial Sequence
  Oligonucleotide
gccgactgaccgaccccacggccgcccgggccccagccca

SEQ ID NO: 4 - DNA, 17 bp, Artificial Sequence
  Primer Oligonucleotide
cgcctggcgcacgcaaa

SEQ ID NO: 5 - DNA, 21 bp, Artificial Sequence
  Primer Oligonucleotide
ttacggtcggggcccgggctc